(19) United States
(12) Patent Application Publication
(10) Pub. No.: US 2023/0107691 A1
(43) Pub. Date: Apr. 6, 2023

(54) Closed Loop System Using In-ear Infrasonic Hemodynography and Method Therefor

(71) Applicants: Anna Barnacka, Cambridge, MA (US); Jal Mahendra Panchal, Boston, MA (US)

(72) Inventors: Anna Barnacka, Cambridge, MA (US); Jal Mahendra Panchal, Boston, MA (US)

(21) Appl. No.: 17/960,815

(22) Filed: Oct. 5, 2022

Related U.S. Application Data
(60) Provisional application No. 63/252,519, filed on Oct. 5, 2021.

(51) Int. Cl. A61B 8/08; A61B 8/12; A61B 8/02; A61B 8/04; A61B 5/00; G06N 5/02

(52) U.S. Cl. CPC A61B 8/5223; A61B 8/12; A61B 8/02; A61B 8/04; A61B 8/0883; A61B 5/486; G06N 5/022

(57) ABSTRACT

A closed loop system using in-ear infrasonic hemodynography and method therefor are disclosed. The system includes an in-ear biosensor system that detects biosignals including infrasonic signals of an individual, and sends the biosignals to an analysis system that identifies physiological data from the biosignals that is associated with the autonomic nervous system of the individual. External sensors can detect other physiological data of the individual during environmental conditions and under different stimuli, and send the other data and the context under which it was detected to the analysis system. The analysis system can train a machine learning model with the identified physiological data in conjunction with the other physiological data, execute actions in response to new information to adjust the autonomic nervous system of the individual, optimize their performance on tasks, and train the individual to adjust their autonomic nervous system in response to new stimuli.

### RELATED APPLICATIONS

This application claims the benefit under 35 USC 119(e) of U.S. Provisional Application No. 63/252,519 filed on Oct. 5, 2021, which is incorporated herein by reference in its entirety.

### BACKGROUND OF THE INVENTION

Biometrics refers to processes and systems for obtaining and analyzing biological measurements and physical and behavioral characteristics of individuals. Biometrics systems obtain and analyze the biological measurements and characteristics, which are also known as biometric data.

Biometric data monitoring is crucial to understanding health and diseases. Interest in this area monitoring has grown recently, particularly due to the increasing cost of healthcare, prolonged life expectancy, recent pandemics, and advancements in wearable technology.

Physiological processes of individuals such as respiratory rate, heart rate, blood pressure, muscle activity, sweat gland activity and internal movements of organs generate electric, thermal, chemical and acoustic energy. These generated energies are also known as biosignals. Biometrics systems can detect these biosignals using detector/transducer devices (“detector devices”) of various technologies attached to different parts of the body, These devices include: wearable and wireless devices, smartphone-connected technologies, implantable sensors and various lab-on-a-chip nanosensor platforms, in examples.

The detector devices of the biometric systems detect the biosignals and generate signals representing aspects of the biosignals. The generated signals can be in the form of electric potential, pressure difference, mechanical vibrations or acoustic waves, in examples. The generated signals form sets of biosignal data. The biometric systems then analyze the biosignal data, and measure/quantify aspects of and changes to the data over time to obtain the biometric data. In the case of the aforementioned physiological processes, the biometric data obtained and measured are also known as cardiovascular measurements. For brevity, the generated signals that form the sets of biosignal data are hereinafter referred to simply as “biosignals.”

Historically, cardiologists have used contemporary biometrics systems to obtain the cardiovascular measurements in both clinical settings and remote monitoring settings. The contemporary biometrics systems include catheter systems and electrocardiogram (ECG) systems, in examples. In particular, the ECG systems set a standard for measurement accuracy of cardiovascular measurements such as heart rate (HR), inter-beat interval (IBI), and heart rate variability (HRV). HR is a measure of average beats per minute, while HRV is typically expressed in milliseconds and measures the changes in time, or variability, between successive heartbeats/IBIs.

The contemporary biometrics systems have limitations. They require in-person visits to a clinic or hospital, are expensive and invasive. The catheter systems, in one example, require that a technician or other medical professional insert a catheter into the individual's artery. The ECG systems, in another example, require placement of multiple electrodes connected to wires upon the individual's skin at or near the heart and major arteries.

The autonomic nervous system is a control system of the body that acts largely unconsciously. The autonomic nervous system regulates many of the aforementioned physiological processes and other bodily functions such as digestion, pupillary response, urination, and sexual arousal.

The autonomic nervous system has two basic portions, a sympathetic nervous system and a parasympathetic nervous system. The sympathetic nervous system dominates during moments of stress, physical activity and when the individual is in danger. For these reasons, the sympathetic nervous system is often associated with a “fight-or-flight” response. The parasympathetic nervous system, in contrast, dominates during periods of rest, during digestion and calmness. For these reasons, the sympathetic nervous system is often referred to as the“rest and digest” portion of the autonomic nervous system.

Biofeedback is a mind-body technique that uses various detector devices to detect biosignals associated with physiological processes of individuals and to detect behaviors exhibited by the individuals in response to an event or stimuli. The technique then allows the individuals to create conscious control over their physiological processes and the behaviors based upon the detected information. The behaviors can include eye movements, changes to body position and posture, and tensing of muscles, in examples. The main goal of biofeedback is self-regulation of your physiological state.

Biofeedback systems collect the information detected during biofeedback over time, and can execute actions in response to the detected information. The actions can include: sending notifications or recommendations for the individuals to change their behavior; informing individuals about changes to their physiology to build self-awareness of their physiology, and to suggest actionable steps to change the physiology; presenting descriptions of the changes or plots of biosignals associated with the changes to a visual display or for audible playback; and making changes directly to the environment around or otherwise perceived by the individuals, in examples. By detecting changes in real-time, and either enabling the individual to make real-time adjustments to their physiological processes and behaviors or making changes directly to the environment perceived by the individuals, the biofeedback systems can provide individuals with a level of conscious control over their autonomic nervous system and body that they typically would otherwise not have.

A closed-loop system measures, monitors, and controls a process. One way in which a process can be accurately controlled is by monitoring its output and “feeding” at least some of the output back as input to the same process. The new output that results from applying new input and the previous output as input to the process can be compared to a desired output, so as to reduce the error of the system. Additionally, if the output begins to diverge from the desired output, also known as creating a disturbance of the system, the input can be adjusted to bring the output of the system back to the original or desired response. The quantity of the output being measured is called the “biofeedback signal,” and the type of control system which uses biofeedback signals to both control and adjust itself is called a closed-loop system.

### SUMMARY OF THE INVENTION

More recently, consumer wearable devices (“wearables”) have emerged as components of biometric systems. The biometric systems that utilize or include the wearables are also known as wearable systems. In the wearable systems, detector devices of the wearables generally send signals representing the aspects of the biometric data they detect to a mobile phone or other wireless device for local processing and analysis. The mobile phone or other wireless device then might send the analyzed information for storage to a remote database. In some wearable systems, the wearables send the detected information for later (in non real-time) analysis to a server in a remote network.

Existing wearables can detect at least some of the cardiovascular measurements using various detector devices of different technologies. These technologies include electric potentials (ECG), photoplethysmography (PPG), oscillometry, biochemical sensors, or a combination of these technologies . Because the wearables are typically worn on the person's wrist or finger and do not require an office visit to operate, the wearable systems are generally more accessible, convenient and less expensive than the contemporary biometric systems.

The technologies used in the detector devices of the existing wearable systems are constantly expanding, and the systems themselves are increasingly using advanced computational approaches to process the signals sent from the devices. As a result, wearable systems are challenging the contemporary biometric systems for their ability to obtain physiological measurements such as cardiovascular measurements, and are increasingly changing how at least some diseases are detected and monitored. This is because the detector devices of the wearables are able to detect and record representations of many different signals, including brain activity (EEG), blood pressure, respiration, and muscle biosignals (EMG), in examples.

However, the existing wearables and their wearable systems have limitations. Generally, the detector devices detect incomplete versions of the energy/phenomena generated by the individuals. This leads to errors when the processing systems of the wearable systems convert the signal representations of the detected information into the cardiovascular measurements. In many cases, the wearable systems are inaccurate with aggregated errors of up to 10 percent in reporting HR, in one example. Additionally, design constraints, including limitations in power consumption, memory usage, and data storage, impact the ability of the wearables to provide precise beat-to-beat assessment. For example, many wearables provide only time-averaged HR measurements over intervals of five (5) minutes or more. Still other existing wearables claim the ability to acquire or record HR data in real-time, such as continuously over time periods on the order of hundreds of milliseconds, but have no local processing ability. Instead, these wearable systems send the data to a remote server over a period of minutes or possibly hours, and the remote server then processes/analyzes the data.

Despite the ability of biometric systems including wearables to generate increasingly large quantities of electronic health data, health care continues to be reactive, treating a disease only after it is diagnosed. This approach narrows the capacity of health care professionals and policy makers to implement preventive measures and assumes that most if not all individuals mimic the common trends of disease trajectories and treatments.

These existing biometric systems including wearables have additional limitations. In one example, the wearables do not provide information regarding the individual in real-time (i.e., on the order of at least seconds, and preferably on the order of hundreds of milliseconds). Such real-time data is critical to characterizing and identifying changes to an individual's autonomic nervous system, which can change in response to stimuli on the order of seconds or even hundreds of milliseconds. In another example, the existing biometric systems do not create individualized baselines of autonomic nervous system behavior of individuals over time, such as days, weeks or even months. Such a corpus of information for each individual obtained over time is key to interpreting the physiological state of the individuals at a given time.

A novel closed loop system is proposed that overcomes the limitations of the existing biometrics systems that include wearables/wearable systems, while providing accuracy that rivals that of the contemporary biometric systems. The closed loop system uses data associated with physiological processes of individuals, and possibly data associated with behavioral characteristics of the individuals, in examples.

The closed loop system utilizes in-ear infrasonic hemodynography (IH) technology that combines the precision and full range biometric data access capabilities of the contemporary biometric systems, with the convenience and low cost of the wearable systems and their wearables. For this purpose, the closed loop system includes a familiar in-ear headphone system that has been adapted to passively detect biosignals. The biosignals are in the form of acoustic signals including infrasonic signals generated by blood flow and other vibrations related to body activity/physiological processes of the individual. The in-ear headphone system is also known as an in-ear biosensor system.

The in-ear biosensor system can detect and collect a continuous stream of biosignals and transmit the biosignals to a mobile device and online server systems in real time. A data analysis system of the closed loop system can then identify and extract physiological data of the individual from the biosignals, where the physiological data includes the various cardiovascular measurements of the individual, and other information identified within the biosignals. In examples, the data analysis system might be located in one or more of the following that are in communication with the in-ear biosensor system: a local area network, a mobile phone, and a remote network/cloud-based network.

By using online servers, the closed loop system is able to perform continuous, real-time data collection and analysis without the problems of battery usage, storage and complex computations related to big data. The closed loop system also allows for instantaneous/real-time analysis and quality assessment of the biosignals, thereby enabling true closed-loop biofeedback capabilities that are not provided by existing biometric systems including wearables and wearable systems.

Experimentation has shown high waveform fidelity for individuals using the closed loop system and its in-ear biosensor system. The closed loop system provides a 0.99 correlation in HR and IBI measurements as compared to the “gold standard” ECG systems. Thus, the proposed closed loop system is the first demonstration of IH capabilities that can deliver accuracy comparable to ECG systems, where the detector devices are in a wearable form factor.

The in-ear biosensor system has multiple design advantages such as familiar form factor, multipurpose use and low battery usage. The IH signals show high fidelity within subjects allowing for accurate measurements of body vitals when compared with standard methods of measurement. The continuous data stream from the IH earbuds provides detailed information on time scales as short as milliseconds, generating more than 2.8 MB of data per hour. The in-ear biosensor system allows for continuous monitoring without compromising data quality and sampling rate.

In general, according to one aspect, the invention features a closed loop system. The system includes an interface configured to receive biosignals including infrasonic signals from an in-ear biosensor system worn by an individual, and a data analysis system that monitors the received biosignals at the interface over time and identifies physiological data of the individual based upon the received biosignals.

The data analysis system creates a baseline autonomic nervous system profile of the individual over a time period from the identified physiological data, and the baseline autonomic nervous system profile tracks changes to a physiological state of the individual over the time period. The data analysis system also identifies current physiological data of the individual from new biosignals received at the interface over a current time period, and identifies a current physiological state of the individual by mapping the current identified physiological data against the baseline autonomic nervous system profile.

In examples, the physiological data includes a heart rate, a heart rate variability, a blood pressure measurement, a respiration rate, a stroke volume and a heart contractility of the individual. In one implementation, the data analysis system creates a baseline autonomic nervous system profile over a time period by plotting one or more types of the identified physiological data against one or more other types of the physiological data.

In another implementation, the data analysis system creates a baseline autonomic nervous system profile of the individual over a time period by passing the identified physiological data to a machine learning model for training, where the trained machine learning model incorporates the baseline autonomic nervous system profile of the individual. Thee data analysis system then maps the current identified physiological data against the baseline autonomic nervous system profile by passing the current identified physiological data as input to the trained machine learning model, the result of which is the current physiological state of the individual.

In yet another implementation, the data analysis system creates the baseline autonomic nervous system profile of the individual from the identified physiological data and from other physiological data received at the interface, where the other physiological data is detected by and sent from one or more external sensors monitoring the individual.

In still another implementation, the data analysis system creates the baseline autonomic nervous system profile of the individual from the identified physiological data and from user provided physiological data received at the interface.

Additionally and/or alternatively, the data analysis system might present the current physiological state of the individual and the baseline autonomic nervous system profile of the individual to the interface for access by one or more external systems.

In another example, when the data analysis system maps the current identified physiological data against the baseline autonomic nervous system profile, if the current identified physiological data deviates from that of the physiological data in the profile by a threshold amount, the data analysis system instructs the individual to perform one or more actions designed to adjust the current physiological state of the individual to be similar to that of the physiological state in the profile.

In yet another example, the data analysis system accesses a target physiological state at the interface that was sent to the interface by a system external to the closed loop system, where the closed loop system instructs the individual to perform one or more actions designed to adjust the current physiological state of the individual to be that of the target physiological state.

In general, according to another aspect, the invention features a method of operation for a closed loop system. The method comprises: receiving, at an interface, biosignals including infrasonic signals from an in-ear biosensor system worn by an individual; monitoring the received biosignals at the interface over time and identifying physiological data of the individual based upon the received biosignals; creating a baseline autonomic nervous system profile of the individual over a time period from the identified physiological data, the baseline autonomic nervous system profile tracking changes to a physiological state of the individual over the time period; and identifying current physiological data of the individual from new biosignals received at the interface over a current time period, and identifying a current physiological state of the individual by mapping the current identified physiological data against the baseline autonomic nervous system profile.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1A is a schematic diagram of an exemplary closed loop system, according to an embodiment;

FIG. 1B is a schematic diagram of another exemplary closed loop system, according to another embodiment;

FIG. 2A through 2C each show: plots of biosignals from an in-ear biosensor system of the closed loop system worn by an individual; ECG signals from an ECG system connected to the same individual; and a tachogram created from these signals, where: FIG. 2A plots the signals and the tachogram during normal breathing; FIG. 2B plots the signals and the tachogram during a breathing exercise that uses resonant breathing; and FIG. 2C plots the signals and the tachogram during a Valsalva maneuver;

FIG. 3A through 3C are power spectra plots for each of the tachograms in FIG. 2A through 2C, respectively;

FIG. 4 is a diagram that shows the basic components of the autonomic nervous system of an individual, where the diagram includes different types of physiological data that the closed loop system can identify, extract, or otherwise obtain from the detected biosignals, and where the diagram also includes examples of other physiological data that sensors external to the closed loop system (“external sensors”) can detect and send to the closed loop system, and where the diagram also illustrates the effect that changes to each type of physiological data generally have upon the autonomic nervous system;

FIG. 5 is an exemplary baseline autonomic nervous system profile of the individual over a time period, where the closed loop system identifies physiological data of the individual from the biosignals and plots one type of the physiological data (here, a heart rate variability) against another type of the data (here, a heart rate) to create the profile;

FIG. 6 is a diagram that shows how exemplary data points from the baseline profile in FIG. 5 translate to different physiological states of the individual's autonomic nervous system;

FIG. 7 is a flowchart that describes a method of operation of the closed loop system;

FIG. 8 is a flowchart that provides more detail for the method of FIG. 7; and

FIG. 9 is a flowchart that describes another method of operation of the closed loop system.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

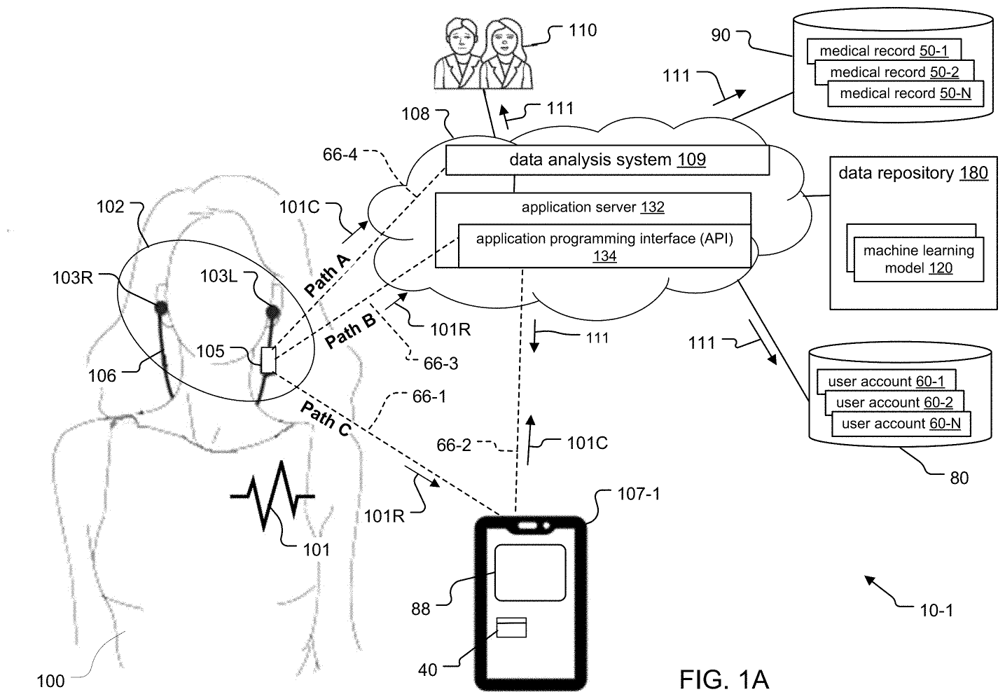

FIG. 1A shows an exemplary closed loop system 10-1. The system 10-1 includes an in-ear biosensor system 102 worn by an individual 100, a user device 107 carried by the individual 100 and various components within and/or in communication with a network cloud 108.

The components within and/or in communication with the network cloud 108 include a data analysis system 109 and an application server 132, a medical record database 90, a user account database 80 and a data repository 180. The medical record database 90 includes medical records 50 of individuals 100, while the user account database 80 includes user accounts 60 of individuals 100 that are authorized users of the system 10. The data repository 180 includes one or more machine learning models 120.

A computing device includes at least one or more central processing units (CPUs) and a memory. The CPUs have internal logic circuits that perform arithmetic operations and execute machine code instructions of applications (“application code”) loaded into the memory. The instructions control and communicate with input and output devices (I/O) such as displays, printers and network interfaces.

The CPUs of the computing devices are typically configured as either microprocessors or microcontrollers. A microprocessor generally includes only the CPU in a physical fabricated package, or “chip.” Computer designers must connect the CPUs to external memory and I/O to make the microprocessors operational. Microcontrollers, in contrast, typically integrate the memory and the I/O within the same chip that houses the CPU.

The CPUs of the microcontrollers and microprocessors of the computing devices execute application code that extends the capabilities of the computing devices. In the microcontrollers, the application code is typically pre-loaded into the memory before startup and cannot be changed or replaced during run-time. In contrast, the CPUs of the microprocessors are typically configured to work with an operating system that enables different applications to execute at different times during run-time.

The operating system has different functions. The operating system enables application code of different applications to be loaded and executed at run-time. Specifically, the operating system can load the application code of different applications within the memory for execution by the CPU, and schedule the execution of the application code by the CPU. In addition, the operating system provides a set of programming interfaces of the CPU to the applications, known as application programming interfaces (APIs). The APIs allow the applications to access features of the CPU while also protecting the CPU. For this reason, the operating system is said to execute “on top of” the CPU. Other examples of CPUs include Digital Signal Processors (DSPs), Application Specific Integrated Circuits (ASICs), and Field Programmable Gate Arrays (FPGAs).

The in-ear biosensor system 102 includes left and right earbuds 103L, 103R and a controller board 105. The earbuds 103 communicate with one another and with the controller board 105 via earbud connection 106. Here, the earbud connection 106 is a wired connection, but wireless connections are also supported. Here, the controller board 105 is external to the earbuds, but the controller board 105 can be also embedded in the earbuds 103L, 103R. One or more of the earbuds also typically includes a speaker that presents audio to the individual.

The user devices 107 include portable user devices and stationary user devices. In examples, the portable user devices include mobile phones, smart glasses, smart watches, and laptops, in examples. The stationary user devices include workstations and gaming systems, in examples. A mobile phone/smartphone user device 107 is shown.

Each user device 107 is a computing device that includes a display 88 and one or more applications. An interactive user application running on each user device 107, or user app 40, is shown. The user app 40 of each user device 107 executes upon a CPU of the user device 107, receives information sent by other components in the system 10 and presents a graphical user interface (GUI) on the display 88. The GUI allows the individual 100 to enter information at the user app 40 and can display various information upon the display 88.

The application server 132 is a computing device that connects the biosensor system 102 and the user device 107 to the components within or at the network cloud 108. The application server 132 includes secure website software (or a secure proprietary application) that executes on the application server 132.

Medical professionals 110 are also shown. The medical professionals 110 include doctors, nurses/nurse practitioners, physician's assistants, and medical technicians, in examples. The medical professionals 110 are trained in the use of the contemporary biometric systems and the closed loop system 10-1. The medical professionals use computing devices such as laptops or smartphones to securely connect to the network cloud 108. In examples, the medical professionals 110 can connect to the network cloud 108 through telehealth services, or virtual clinics, with user 100 information provided by the closed loop system 10-1.

The medical professionals 110, the databases 80/90, the user devices 107 and the data repository 180 can connect to the network cloud 108 and/or components within the cloud 108 in various ways. These connections can be wired Internet-based or telephony connections, wireless cellular connections, and/or wireless Internet-based connections (e.g., Wi-Fi), in examples. In examples, the network cloud 108 can be a public network, such as the Internet, or a private network.

The in-ear biosensor system 102 and the user devices 107 communicate with each other and with the network cloud 108 via one or more wireless communications links 66. In more detail, the user device 107 connects to the in-ear biosensor system 102 via wireless link 66-1 and connects to the application server 132 via wireless link 66-2. The in-ear biosensor system 102 can also communicate with the application server 132 via wireless link 66-3 and might connect directly to the data analysis system 109 via wireless link 66-4. The wireless links 66 might be cellular-based or Internet-based (e.g., IEEE 802.11/Wi-Fi), or possibly even Bluetooth. In one example, the wireless links 66-3 and 66-4 are high-speed 5G cellular links. These links 66 are also encrypted to provide secure communications between the components that are at endpoints of the links 66.

In the illustrated example, the data analysis system 109 and the application server 132 are located in the network cloud 108. The network cloud 108 is remote to the individual 100. In this way, the application server 132 and the data analysis system 109 can service possibly thousands or more individuals 100 that are in different geographically distributed locations. Alternatively, the data analysis system 109 and/or the application server 132 might also be located on a local area network within a premises, such as a residence, commercial building or place of business of the individual 100. In one implementation, the capabilities provided by the application server 132 are incorporated into the data analysis system 109.

Infrasound

Biosignals such as acoustic signals are generated internally in the body by breathing, heartbeat, coughing, muscle movement, swallowing, chewing, body motion, sneezing and blood flow, in examples. The acoustic signals can also be generated by external sources, such as air conditioning systems, vehicle interiors, various industrial processes, etc. The acoustic signals include audible and infrasonic signals.

The acoustic signals represent fluctuating pressure changes superimposed on the normal ambient pressure of the individual's body and can be defined by their spectral frequency components. Sounds with frequencies ranging from 20 Hz to 20 kHz represent those typically heard by humans and are designated as falling within the audible range. Sounds with frequencies below the audible range (i.e., from 0 Hz to 20 Hz) are termed infrasonic or infrasound. The level of a sound is normally defined in terms of the magnitude of the pressure changes it represents. These changes can be measured and do not depend on the frequency of the sound.

The left and right earbuds 103L,103R detect the biosignals 101 from the individual 100 via sensors included within one or more of the earbuds 103. These sensors include acoustic sensors, which can detect sounds in both the infrasonic and audible ranges, vibration sensors and pressure sensors, and possibly dedicated infrasonic sensors, in examples. The sensors of the in-ear biosensor system 102 detect the biosignals 101 and send a representation of the biosignals to the data analysis system 109 for analysis. For brevity, the representation of the biosignals are hereinafter simply referred to as “biosignals.”

The biologically-originating sound detected inside the ear canal by the earbuds 103 is mostly in the infrasound range. In particular, the infrasound and vibration sensors can detect biosignals from the individual 100 that include information associated with operation of the individual's cardiovascular system and musculoskeletal system.

Typically, the biosignals are detected at each of the earbuds 103L,R at substantially the same time. This “stereo effect” can be utilized to identify and address artifacts, as well as improve a signal to noise ratio (SNR) and coverage of the biosignals 101 and thus provide high quality signals for subsequent characterization and analysis. Here, coverage refers to the improved ability of two sensors to detect biosignals of an individual than a single sensor, without gaps in the detected signals due to improper sensor positioning, sensor failure or power loss, in examples.

The closed loop system 10-1 generally operates as follows. An individual enters his/her credentials at the GUI of the user app 40, which the user device 107 sends over link 66-2 to the application server 132. The application server 132 receives the credentials and verifies that the credentials are associated with an authorized user of the closed loop system 10-1. For this purpose, the secure website software at the application server 132 compares the received credentials to those stored within the user accounts 60 of the user account database 80. Upon finding a match, the application server 132 establishes an authenticated, secure login session over wireless connection 66-2 between the user app 40 and the application server 132 for the individual 100 as an authorized user of the closed loop system 10-1.

The earbuds 103L,103R of the in-ear biosensor system 102 continuously detect the biosignals 101 from the individual 100 and send the biosignals to the controller board 105. Here, the biosignals 101 are in “raw” format: they are uncompressed and may include some noise and/or motion artifacts. In another embodiment, the biosignals might also be compressed, filtered, and pre-analyzed. The controller board 105 buffers the biosignals for subsequent secure transmission to the data analysis system 109.

Once the application server 132 indicates to the user device 107 that the individual 100 is an authorized user, the user device 107 signals the controller board 105 to send the biosignals to the data analysis system 109 by way of one or more communications paths. These paths are labeled Path A, B, and C in the figure. These paths respectively include zero, one, or more than one intermediary components or “hops” between the controller board 105 and the data analysis system 109. The decision of whether to send the biosignals 101 along the different paths depends on factors including the CPU speed of the components at the endpoints of the links 66, the buffer sizes of the wireless transceivers in the components that form each path, and characteristics of the wireless links 66 that form the communications paths. These characteristics include speed, level of encryption and available bandwidth, in examples. A description for each Path A, B and C follows hereinbelow.

Path C is typically the slowest communications path. This path includes wireless links 66-1 and 66-2, and includes the user device 107 and the application server 132 as intermediary components between the in-ear biosensor system 102 and the data analysis system 109. In more detail, the controller board 105 first sends raw versions of the biosignals 101R over link 66-1 to the user device 107. The user app 40 then compresses the raw biosignals 101R into compressed versions of the biosignals 101C for transmission over link 66-2 to the application server 132. In one example, the user app 40 sends the biosignals 101C to the application server 132 via its API 134. The application server 132 then decompresses and forwards the biosignals 101 to the data analysis system 109. The biosignals 101 might be time-stamped by the in-ear biosensor system 102 prior to sending the biosignals 101 to the application server 132/interface 134, or the application server 132/interface 134 may provide this function.

Path B is generally faster than Path C. Path B includes wireless link 66-3 and only one intermediary component, the application server 132, between the controller board 105 and the data analysis system 109. Because link 66-3 is a fast or high throughput link (such as a 5G cellular link), the controller board 105 can send the raw biosignals 101R over link 66-3 to the application server 132 without having to compress the data prior to transmission. Here, the application server 132 can perform various operations on the raw biosignals 101R before forwarding the biosignals to the data analysis system 109 for analysis. These operations include filtering and characterization, authentication, and/or buffering of the signals, in examples. In one example, the controller board 105 sends the biosignals 101R to the application server 132 via its API 134.

Path A is typically the fastest path because it utilizes direct link 66-4 to the data analysis system 109. As a result, the in-ear biosensor system 102 can send the raw biosignals 101R directly to the data analysis system 109 via Path A. The biosignals 101 might be time-stamped by the in-ear biosensor system 102 prior to sending the biosignals 101 to the data analysis system 109, or the data analysis system 109 may provide this function.

The data analysis system 109 then analyzes the biosignals 101 and can use information from the data repository 180 during the analysis. For example, the data analysis system 109 can use the machine learning models 120 in conjunction with the biosignals 101 and historical biosignals for the same individual to predict how the individual's HR might change in response to new stimuli. The data analysis system 109 and/or the application server 132 can access and update the medical record 50 of the individual 100 during and in response to the analysis.

The data analysis system 109 can also send various notification messages 111 in response to the analysis of the biosignals 101. The notification messages 111 include information concerning the analysis and the results of the analysis. The messages 111 can be sent to the medical professionals 110, the databases 80/90, the user devices 107, and possibly even the controller board 105 of the in-ear biosensor system 102. The notification messages 111 can be in the form of an email, SMS/text message, phone call, database record in proprietary format or XML or CSV format, or possibly even audible speech or sounds, in examples. The system can also communicate with other systems and devices to for example adjust for environmental factors (examples: lower temperature in the room, light level, noise, music). System can also display visual content like images or videos to trigger users' reactions inducing for example excitement, relaxation, stimulation based on learned patterns of an individual's biosignals.

The data analysis system 109 can also notify the individual 100 both during and after the analysis via the notification messages 111. In one example, the user app 40 receives the notification messages 111 and might present the notification messages 111 at the display 88, or forward the messages 111 over the wireless link 66-1 to the in-ear biosensor system 102. In another example, the messages 111 might be audible sound messages prepared by the data analysis system 109 or sent by the biosensor system 102 to the connector board 105, for subsequent audio presentation at speakers included within the earbuds 103L,103R.

As a result, the closed loop system 10-1 can continuously monitor biosignals 101 including infrasound signals detected by the in-ear biosensor systems 102 worn by different individuals 100. The data analysis system 109 can then identify and characterize aspects of the biosignals 101 generated by and sent from the in-ear biosensor systems 102 in response to detecting the biosignals 101.

The system 10 can also update medical records 50 for each of the individuals 100, report problems/notify medical professionals 110 of likely medical issues found during the analysis, and provide feedback to the individuals 100 during and upon completion of the analysis.

The in-ear biosensor system 102 includes two earbuds 103 and a controller 105 that communicates with mobile devices 107 via wireless technologies such as Bluetooth Low Energy (BLE). Unlike the sensors of the existing wearables such as PPG sensors, which transmit a light source to detect biosignals, the acoustic sensors are passive sensors. As a result, the power required to operate the acoustic/vibration sensors is minimal as compared to the sensors of most wearables.

In another embodiment, the in-ear biosensor system 102 might also include auxiliary sensors. The auxiliary sensors include accelerometers, pressure sensors, gyroscopes, temperature, humidity and noise level sensors, in examples. One or more of these auxiliary sensors are typically included or otherwise incorporated in one or both earbuds 103. Additionally and/or alternatively, one or more of the auxiliary sensors might be external sensors.

The in-ear biosensor system 102 uses BLE to communicate with the mobile devices 107 to extend battery life even when data is being streamed continuously. While the illustrated in-ear biosensor system 102 has wired connections 106 between the electrical components of each earbud 103 and the controller board 105, the connections can also be wireless. Despite including two earbuds 103L,R, the system 102 can provide high quality results even if only one earbud 103 is available.

FIG. 1B shows another closed loop system 10-2. The closed loop system 10-2 includes substantially similar components as the system 10-1 of FIG. 1A, and includes additional components. These additional components include external sensors/systems such as an ECG system 48, a wrist-worn wearable 38 and an eyeglass user device 107-2. The closed loop system 10-2 can also include external sensors/systems such as a virtual reality headset 58 or augmented reality headsets/glasses/screens.

In the illustrated example, the data analysis system 109 receives biosignals not only from the in-ear biosensor system 102, but also receives biosignals (or physiological data obtained from the biosignals) from the wrist-worn wearable 38 and the ECG system 48. In addition, the data analysis system 109 can receive physiological data of the individual 100 detected by and sent from the eyeglass user device 107-2 (here, pupil dilation or eye movement information, in examples). The physiological data can be also obtained from cameras in the VR headset 58, augmented reality headset or other device with a display or monitor that is able to capture images of the individual 100.

The ECG system 48 includes a display 88, electrodes 22 and wires/leads 26 attached to the electrodes 22. The electrodes 22 detect changes in electrical signals associated with cardiovascular activity of the individual 100 over a series of successive heart cycles. Via its wires 26, the ECG system 48 sends a representation of the detected ECG electrical signals (ECG signals 24) to the data analysis system 109 for analysis. While not shown, the ECG system can use wired or wireless links in which to send the data.

The wrist-worn wearable 38 also detects biosignals. The wearable sends biosignals (or physiological data obtained from the biosignals to the user device 107-1, which collects the information to the data analysis system 109. In the illustrated example, the wearable 38 includes an ECG electrode that detects ECG signals 24, but other sensors such as PPG sensors might be used.

The eyeglass user device 107-2 includes an app 40, a display 88 and incorporates a camera 98. Here, the camera 98 can detect physiological data of the individual 100 such as eye movements, facial expressions and posture, and can send the data over wireless link 66-5 to the data analysis system 109 via the application server 132 for analysis.

The VR headset 58, in a similar vein, can obtain image snapshots of video or a sequence of video frames of the individual 100. The VR headset 58 might also preprocess the snapshots or video frames and include metadata with the images and/or frames. The metadata might include bounding boxes created for various facial features and locations, x-y coordinate values for changes in eye position and/or pupil dilation, in examples. The VR headset 58 or other headset then sends the images and/or video frames over wireless link 66-6 to the data analysis system 109 via the application server 132.

It can also be appreciated that the eyeglass user device 107-2 and/or the VR headset 58 can send their information directly to the data analysis system 109, via a high-speed wireless link such as link 66-4 for Path A.

The in-ear biosensor system 102 shares a similar architecture to many consumer wireless Bluetooth in-ear headphones and can be used simultaneously to collect IH signals and play audio. Each earbud 103 has an integrated acoustic sensor that enables the measurement of small variations in in-ear acoustic pressure. The turbulence associated with the heart sounds and vascular hemodynamics have specific infrasound features that are captured by the earbuds 103.

While IH signals are captured below the range of human hearing (<20 Hz), audio output is conventionally restricted to within the range of human hearing (20 Hz to 20 kHz); thus, there is minimal interference in the IH signal when speaker audio is present. This phenomenon allows for novel methods of physical acoustical tuning, which contribute to an earbud design that optimizes IH signal acquisition and preserves audio quality comparable to that of consumer-grade in-ear headphones. Thus, health monitoring can be performed in the background when a person is wearing in-ear headphones during daily activities like, for example, listening to music or taking calls. Such seamless integration with a personal lifestyle can provide a significant data stream, which combined with real-time analytics provides a platform to build applications that could lead to improvement in health outcomes.

Biosignal Sensitivity

The IH technology of the in-ear biosensor system 102 enables continuous monitoring of multiple vital functions. In order to assess sensitivity of the closed loop system and its ability to detect physiological data of the individual , participants of a study were asked to perform different breathing maneuvers including regular breathing, resonant breathing, and the Valsalva maneuver.

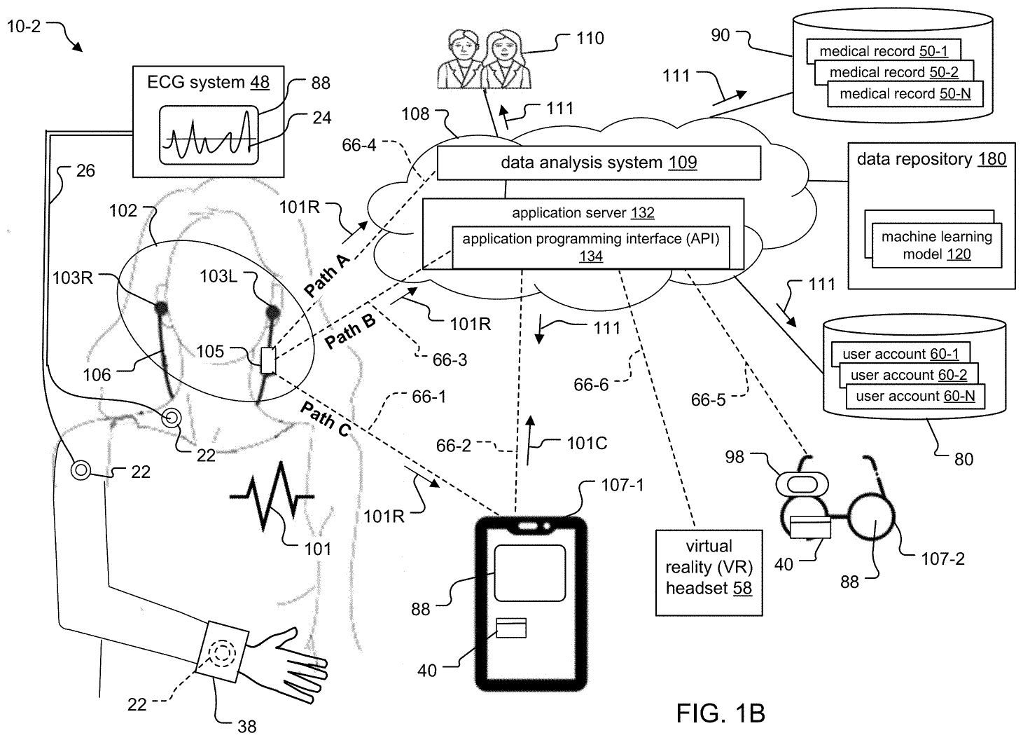

FIG. 2A through 2C each plot biosignals 101, ECG signals 24 and tachograms 402 of an exemplary study participant of the closed loop system 10 over a 60 second period. The plots show significant changes in IBI patterns, and are reflective of changes in physiology demonstrated with ECG and IH biosignals 101 during regular breathing (FIG. 2A), resonant breathing (FIG. 2B), and Valsalva maneuver (FIG. 2C).

For each of FIG. 2A-2C, tachograms were computed, and HR and HRV values were obtained and averaged to assess sensitivity of IH IBIs to provide biofeedback and compare results to ECG. Additionally, a custom peak-detection algorithm was employed to detect ECG R peaks, which were also visually verified to ensure no artifacts are present in the reference ECG dataset. ECG RR intervals were calculated from successive R peaks and were used to construct the tachograms and calculate the average HR and HRV. Such a processing pipeline is used to determine data quality, identify peaks, calculate IBI, HR, HRV and tachograms for IH data. HR and HRV are calculated within 5-second intervals.

Each of the plots include biosignals 101 detected from the in-ear biosensor system 102 worn by an individual, ECG signals 24 detected by an ECG system 48, and tachograms 402 created from the biosignals 101 and the ECG signals. The amplitudes of the biosignals 101 and ECG signals 24 are expressed in volts, and the amplitudes of the tachograms 402 are expressed in inter beat times/IBIs. The tachograms 402 provide an HR measurement in beats per minute (BPM), and HRV measurement can also be obtained. The tachograms created from the ECG are plotted as squares, while the tachograms created from the biosignals 101 are platted as red circles.

The changes in tachograms over the span of a breathing cycle reflect the balance between sympathetic and parasympathetic nervous systems. Different breathing techniques induce varying degrees of changes in beat-to-beat variations and hemodynamics that are the response of the autonomic nervous system. Respiratory sinus arrhythmia during breathing, as well as changes in HRV related to stress are indicators of this balance. Data with excessive noise are denoted by a gray background in the IH signal/biosignals 101.

In more detail, the signals plotted during the regular breathing pattern shown in FIG. 2A illustrates the nominal baseline changes in physiology and low levels of heart rate variability that typically occur during at-rest regular breathing. The average HR measured with both IH and ECG is 84.7 BPM. The HRV measured with IH is 2.6 BPM (22 ms) and 2.0 BPM (17 ms) measured with ECG. Both HR and HRV are relatively constant during data collection. Such relatively low HRV is typical for the sympathetic nervous system response.

Resonant breathing is a breathing technique that maximizes respiratory sinus arrhythmia (RSA), the change in IBI or HR related to breathing. Breathing exercises with specific inhale:exhale ratios lead to resonant breathing and can induce large amplitude sinusoidal patterns in the RSA. In the example shown in FIG. 2B, a change in IBI of about 300 ms (35 BPM) is observed during resonant breathing, while the averaged heart rate changes by about 7 BPM.

The Valsalva maneuver is a way to transiently increase intrathoracic pressures and is commonly performed by moderately forceful exhalation against a closed airway. This method leads to dramatic changes in the systemic blood pressure and HR that the autonomic nervous system attempts to compensate for and correct (see Reference 28). In the illustrated example, with reference to FIG. 2C, the subject performed the bearing down method to induce the Valsalva maneuver. For the breath hold duration, IBI values decreased by over 300 ms and rebounded by nearly 400 ms at the end of the maneuver. The amplitude of the IH signal followed the same pattern as the IBI, with ˜30% drop followed by an ˜80% increase.

The beat-to-beat variations in IBI are clearly visible in the tachograms for patterns or exercises that induce large variability, in particular, the resonant breathing exercises and the Valsalva maneuver. Overall, breathing affects HRV with IBI changing up to 300 ms for different breathing techniques. On the other hand, variability in IBI is muted for regular breathing. For all cases, the tachogram derived from IH captures IBI changes at short timescales.

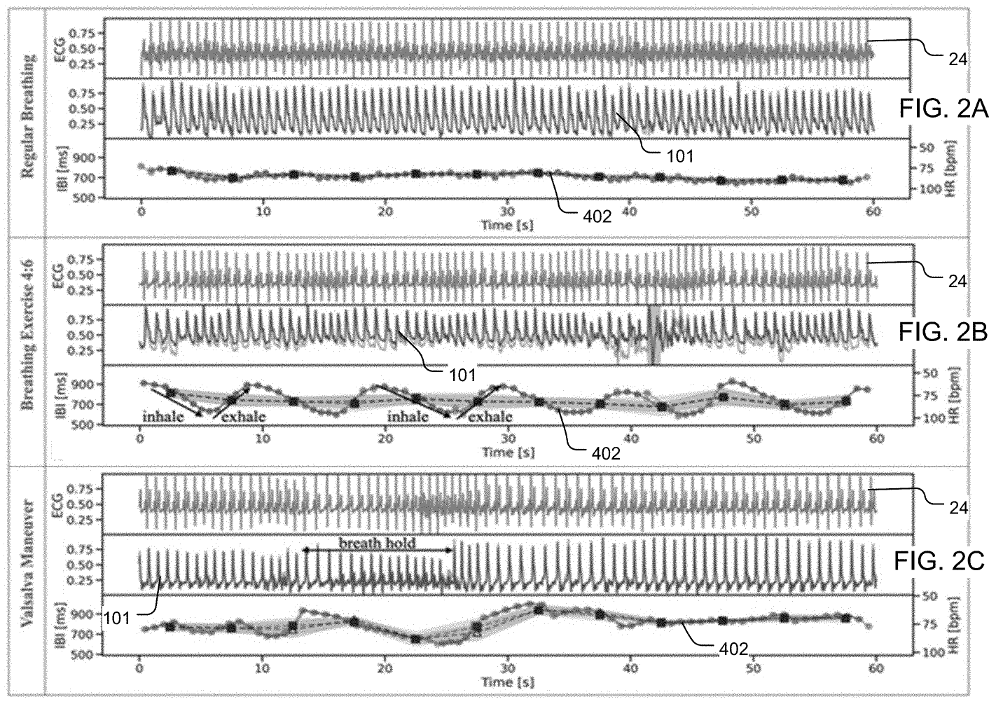

FIG. 3A through 3C are power spectra plots for each of the tachograms in FIG. 2A through 2C, respectively. In each of FIG. 3A through 3C, the low-frequency band and the high frequency band are indicated via a legend. The frequency domain representation of the biosignals is indicated by reference 101′. Each of the plots is in units of normalized power versus frequency in Hz.

The locations of spectral peaks from the tachograms in FIG. 3A-3C correspond to the respiratory rate expected from each breathing pattern. For example, fundamental breathing frequencies for a 4-second inhale and a 6-second exhale pattern (4:6 pattern) was 0.1 Hz indicating a breathing rate of 6 breaths per minute. The low-frequency (LF) band and the high-frequency (HF) band marked on the power spectra are defined as the integrated power within 0.04-0.15 Hz and 0.15-0.4 Hz respectively. Under controlled conditions, the integrated power in the LF and HF bands can be used to estimate the ratio between sympathetic and parasympathetic nervous system activity and can also distinguish controlled breathing from spontaneous breathing. Comparison between metrics calculated using IH signals/biosignals 101 and ECG signals 24 show that the in-ear biosensor system 102 is capable of continuously monitoring body vitals and providing accurate feedback. LH and HF, as well as their ratios can be used as additional input to characterize the state of the autonomic nervous system.

Existing wearables have many drawbacks that limit their use as reliable sources for health monitoring. In one example, consumer wearables have high aggregate errors (up to 10%) in calculating vital measurements due to discrepancy in sampling methods, proprietary algorithms used and quality of data. In another example, there are intrinsic differences between methods used by different wearables for calculating cardiac activity. For instance, PPG devices in smartwatches typically use the time differences between two peaks, known as the P-P intervals, recorded at a low sampling rate (50 Hz) to quantify HR. Errors in localizing peaks due to sampling inaccuracies alone can vary measurement P-P intervals by 50 ms. Additionally, many wearables monitor cardiac activity intermittently or report only processed biometrics that are often calculated from signal averages. By ignoring beat-to-beat variations, these algorithms fail to accurately identify rhythm disturbances such as atrial fibrillation that can be indicative of underlying cardiovascular conditions.

In contrast, the closed loop system 10, through use of its in-ear biosensor system 102, and auxiliary sensors in other embodiments, allows for a precise beat-to-beat cardiac assessment. The metrics presented here might also be combined with advanced processing techniques for early detection of cardiac dysfunction. Since the acoustic/vibration sensors of the in-ear biosensor system 102 can also capture infrasounds generated from multiple systems in the body, IH technology may further be expanded toward comprehensive monitoring of the cardiovascular system as well as other vital functions like respiratory system or brain activity. Additionally, methods of monitoring autonomic nervous system response through IBI and tachograms can be extended towards application in closed-loop biofeedback (e.g., sleep and stress monitoring).

The acoustic/vibration sensors used for IH are capable of capturing infrasonic signals that propagate to the ear canal from various sources within the human body. Here, though the scope of biosignal detection is limited to the observation of pressure waves propagating from the cardiovascular system and into the inner ear canal, these sensors can also pick up speech and bodily motion, even as small as ears twitching and eyes blinking. It is speculated, though, that future studies may reveal use cases for the speech and motion signals captured by the acoustic/vibration sensors, such as assessments of the pulmonary system and structural health of bones.

The closed loop system 10 leverages cloud infrastructure for long-term storage of raw biosignals 101 sent from the in-ear biosensor system 102. Such a system enables studies of trends in existing and new measures for healthcare and other applications. In addition, the novel nature of the IH-based in-ear biosensor system 102 and technology requires flexibility in data management. Retaining access to the raw signal makes it possible to retroactively calculate any new clinically relevant measures that may emerge in future studies.

Data acquired using the IH in-ear biosensor system 102 is sent to a mobile device 107 through BLE, in one example. The mobile device 107 formats the raw biosignals 101 and sends the biosignals to the cloud infrastructure 108 using a secured communication protocol, such as MQTT (Message Queue Telemetry Transport). Next, the data are stored and processed in real-time via the data analysis system 109. The cloud infrastructure 108 enables storage of raw data in large quantities beyond the memory capabilities of the user devices 107 and also allows the closed loop system 10 to scale as numbers of individuals/subscribers increases.

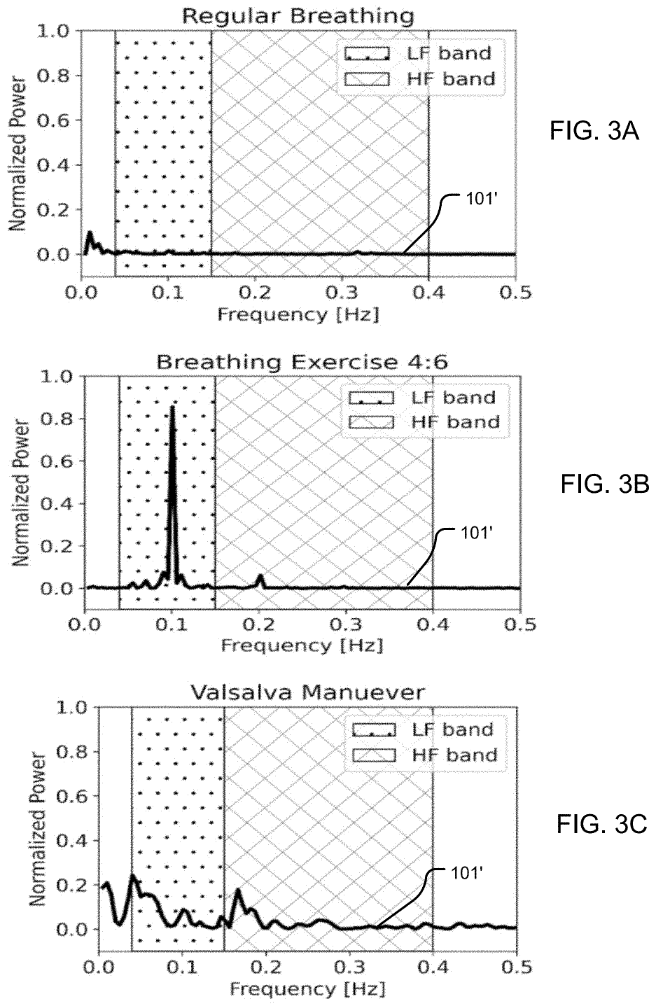

FIG. 4 is a graphic that shows the basic components of the autonomic nervous system 400 of an individual 100. The graphic includes examples of physiological data 410 that the data analysis system 109 can obtain from the biosignals 101 detected by and sent from the in-ear biosensor system, and also includes physiological data 410 that external sensors can detect and send to the data analysis system.

In the illustrated example, different types of physiological data 410 are shown as having an effect on the autonomic nervous system 400. These types include: heart rate 410-1, heart rate variability 410-2, respiration rate 410-3, blood pressure 410-4, and aortic stiffness 410-5. Other types include arterial age 410-6, stroke volume 410-7, heart contractility 410-8, motion 410-9, swallowing 410-10, body temperature 410-11 and pupil diameter 410-12.

Arrows in the diagram also illustrate the “seesaw” nature of each type of physiological data 410 and how changes to each type affect the autonomic nervous system 400. In one example, as the heart rate 410-1 increases in value, the physiological state of the individual 100 tends to adjust more towards alertness/the sympathetic nervous system. This is represented by an “up” arrow placed in the sympathetic nervous system portion in the figure. Alternatively, as the heart rate 410-1 decreases, a shaded “down” arrow is shown in the parasympathetic nervous system portion, indicating that a decrease in the heart rate 410-1 correspondingly tends to adjust the physiological state of the individual more towards the parasympathetic nervous system portion (i.e., the individual is more calm).

In a similar vein, up/down arrows for each of the other types of physiological data 410-2 through 410-12 are also shown. Of these types, increases in the heart rate 410-1, respiration rate 410-3, blood pressure 410-4, aortic stiffness 410-5, arterial age 410-6, heart contractility 410-8, motion 410-9, swallowing 410-10, and the body temperature 410-11 generally cause the physiological state of the individual to adjust more towards the sympathetic nervous system portion (more alert), while increases to the heart rate variability 410-2, the stroke volume 410-7, and the pupil diameter 410-12 generally cause the physiological state of the individual to adjust more towards the parasympathetic nervous system portion (more calm).

The closed loop system 10-1 of FIG. 1A, when it includes no auxiliary sensors, can identify all of these types from the biosignals 101 detected by and sent from the earbuds 103 with the exception of the motion 410-9, body temperature 410-11 and pupil diameter 410-12 physiological data. When the system 10-1 includes the auxiliary sensors, the system 10-1 can detect/identify all types except the pupil diameter 410-12.

In contrast, the closed loop system 10-2 of FIG. 1B can detect/identify all of these types. In one embodiment, when the system 10-2 includes no auxiliary sensors, the system 10-2 can detect/identify the motion 410-9, the body temperature 410-11 and pupil diameter 410-12 via external sensors. In another embodiment, when the system 10-2 includes the auxiliary sensors, the system 10-2 can detect/identify the motion 410-9, the body temperature 410-11 via the auxiliary sensors and the pupil diameter 410-12 via external sensors.

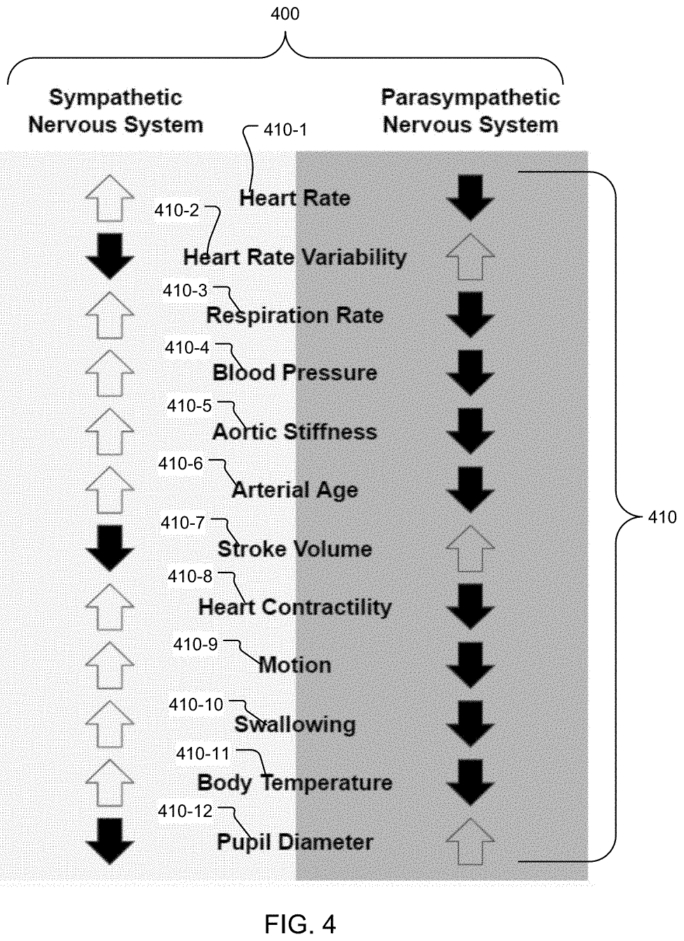

FIG. 5 is an exemplary baseline autonomic nervous system profile 502 of the individual 100 over a time period. Here, the data analysis system 109 identifies physiological data 410 of the individual 100 from the biosignals 101 and plots one type of the physiological data (here, the heart rate variability 410-2) against another type of the data (here, the heart rate 410-1) to create the profile 502. In the illustrated example, the time period over which the data was collected and from which the profile 502 was created is between 10 am and 11 am, over four successive days.

A combination of different physiological data can be used to determine which portion of the autonomic nervous system 400 (sympathetic or parasympathetic) is active for a specific duration of time as compared to the baseline of the individual 100. For example, activation of the parasympathetic nervous system results in decrease of heart rate and increase in heart rate variability while the activation of the sympathetic nervous system results in increase of heart rate and decrease in heart rate variability.

Once a baseline of physiological data for an individual is established, new physiological data can be compared to the baseline to identify which portion of the autonomic nervous system is more active.

Three exemplary points in the profile 502 are also shown. These points are indicated by a square, a circle and a star in the profile 502.

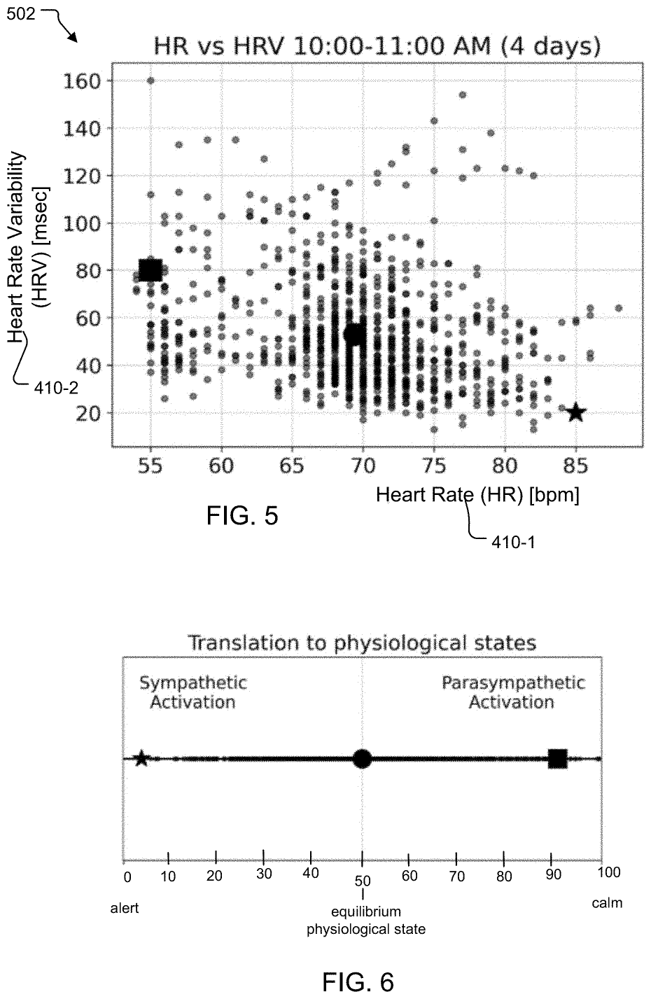

FIG. 6 shows how the three exemplary data points from the baseline profile in FIG. 5 translate to different physiological states of the individual's autonomic nervous system 400. With reference to FIG. 5, the point indicated by the square corresponds to a HRV 410-2 of 80 and a HR 410-1 of 55, the combination of which in FIG. 6 translates to a very calm physiological state. Similarly, the point indicated by the circle in FIG. 5 corresponds to a HRV 410-2 of 55 and a HR 410-1 of 69, the combination of which in FIG. 6 translates to a homeostatic or equilibrium physiological state. Finally, the point indicated by the star in FIG. 5 corresponds to a HRV 410-2 of 20 and a HR 410-1 of 85, the combination of which in FIG. 6 translates to a very alert physiological state.

The figure also shows that the physiological state of the individual 100 is along a continuum, where the physiological state has different degrees or ranges. For this purpose, an arbitrary numerical scale from 0 to 100 is shown, where “0” indicates the most alert, “50” indicates that the physiological state is in equilibrium, while “100” indicates the most calm. By assigning numerical values to the physiological states along the continuum, the closed loop system 10 can determine whether an individual's physiological state is within a range of values, above or below one or more threshold values associated with the physiological state, and possibly make adjustments to the physiological state in light of these values and ranges.

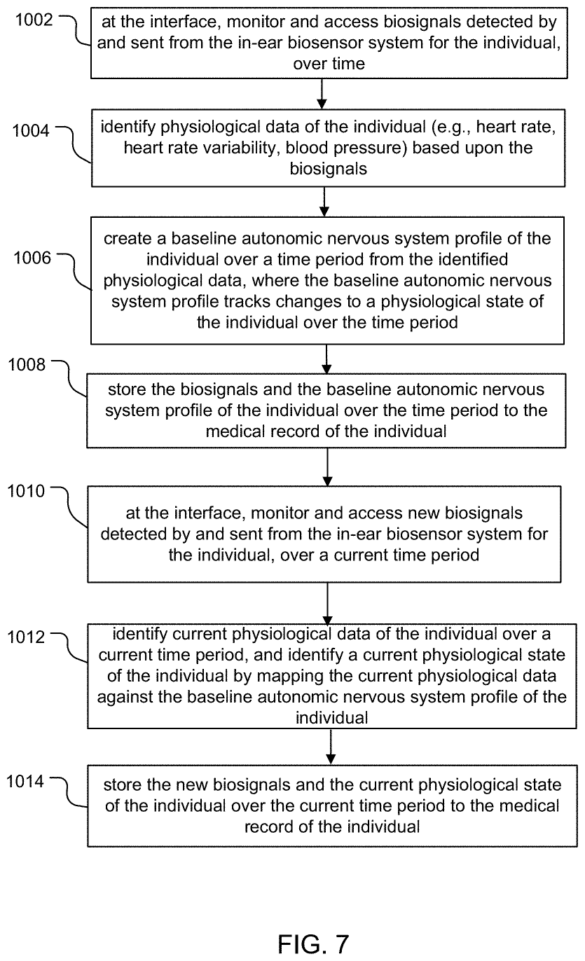

FIG. 7 is a flowchart that describes a method of operation of the data analysis system 109 of the closed loop system 10. The method begins at step 1002.

At step 1002, the data analysis system 109 monitors and accesses biosignals 101 at the API 134. The biosignals 101 are detected by and sent from the in-ear biosensor system 102 work by the individual 100. According to step 1004, the data analysis system 109 identifies physiological data 410 of the individual 100 (e.g., heart rate 410-1, heart rate variability 410-2, blood pressure 410-4) based upon the biosignals 101.

In step 1006, the data analysis system 109 creates a baseline autonomic nervous system profile 502 of the individual 100 over a time period from the identified physiological data 410, where the baseline autonomic nervous system profile 502 tracks changes to a physiological state of the individual over the time period. In examples, the time period can be over days, weeks, months, or possibly for a specific time period each day for a series of days. In one example, the time period might be evening hours (e.g., 8 pm to 11 pm) over a successive number of days. As a result, it can be appreciated that different profiles of the individual can be created for different purposes. Profiles can also be created to capture user circadian rhythms and other body rhythms.

In step 1008, the data analysis system 109 stores the biosignals 101 and the baseline autonomic nervous system profile 502 of the individual over the time period to the medical record 50 of the individual 100. Then, in step 1010, the data analysis system 109 monitors and accesses new biosignals 101 detected by and sent from the in-ear biosensor system 102 for the individual at the interface 134, over a current time period. Preferably, the current time period is shorter than the time period over which the one or more profiles 502 were created.

According to step 1012, the data analysis system 109 identifies current physiological data of the individual over a current time period, and identifies a current physiological state of the individual 102 by mapping the current physiological data against the baseline autonomic nervous system profile of the individual 102. In one example, the data analysis system 109 might pass current physiological data of the individual obtained over a matter of 1-3 minutes, and map it against the baseline profile 502 in FIG. 5. To obtain the current physiological state of the individual, because only one type of physiological data (here, heart rate availability 410-2) is plotted as a function of only one other type of the physiological data (here, heart rate 410-1), the data analysis might 109 perform the mapping by simple linear interpolation of the current physiological data with reference to the identified physiological data in the profile 502 of FIG. 5.

In one implementation, the closed loop system 10, via the data analysis system 109, might map the current identified physiological data of the individual 100 against the baseline autonomic nervous system profile 502 of the individual 100. If the current identified physiological data deviates from that of the physiological data in the profile by a threshold amount, the data analysis system 109 might instructs the individual 100 to perform one or more actions designed to adjust the current physiological state of the individual to be similar to that of the physiological state in the profile.

Additionally, methods like linear and kernel principal component analysis, linear discriminant analysis, single value decomposition, multidimensional scaling, histogram projection, and other machine learning and deep learning methods can be used to translate the physiological data to the current state of the individual 100.

In other examples, the mapping is more complex. Multiple combinations of two or more different types of physiological data can be used to develop a set of profiles using methods like principle component analysis, linear discriminant analysis, single value decomposition, multidimensional scaling, and other machine learning and deep learning methods. These profiles can be used to provide a general or detailed understanding of the physiological state of an individual 100. In step 1014, the data analysis system 109 stores the new biosignals 101 and the current physiological state of the individual over the current time period to the medical record 50 of the individual 100.

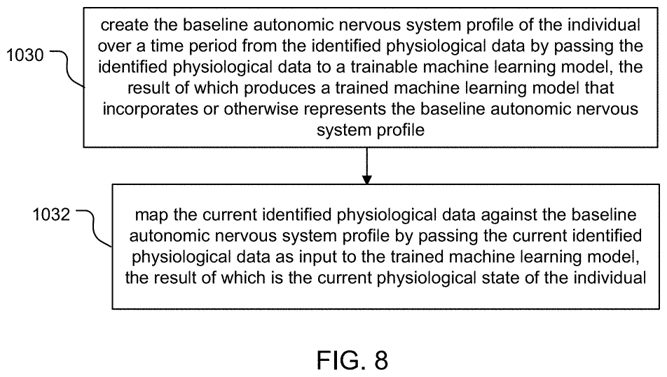

FIG. 8 is a flowchart that provides more detail for the method of FIG. 7. Step 1030 provides more detail for step 1006 in FIG. 7, while step 1032 provides more detail for step 1012 in FIG. 7.

In one implementation, at step 1030, the data analysis system 109 creates the baseline autonomic nervous system profile 502 of the individual 100 over a time period from the identified physiological data by passing the identified physiological data to a trainable machine learning model. The result of this operation produces a trained machine learning model that incorporates or otherwise represents the baseline autonomic nervous system profile 502. In examples, the machine learning model might include or otherwise employ machine learning algorithms including linear regression, decision trees, random forest, XGBoost, Back

Propagation Neural Network and/or deep learning algorithms. Any or all of each might be supervised or unsupervised.

In step 1032, the data analysis system 109 maps the current identified physiological data against the baseline autonomic nervous system profile 502 by passing the current identified physiological data as input to the trained machine learning model. The result of this process is the current physiological state of the individual 100.

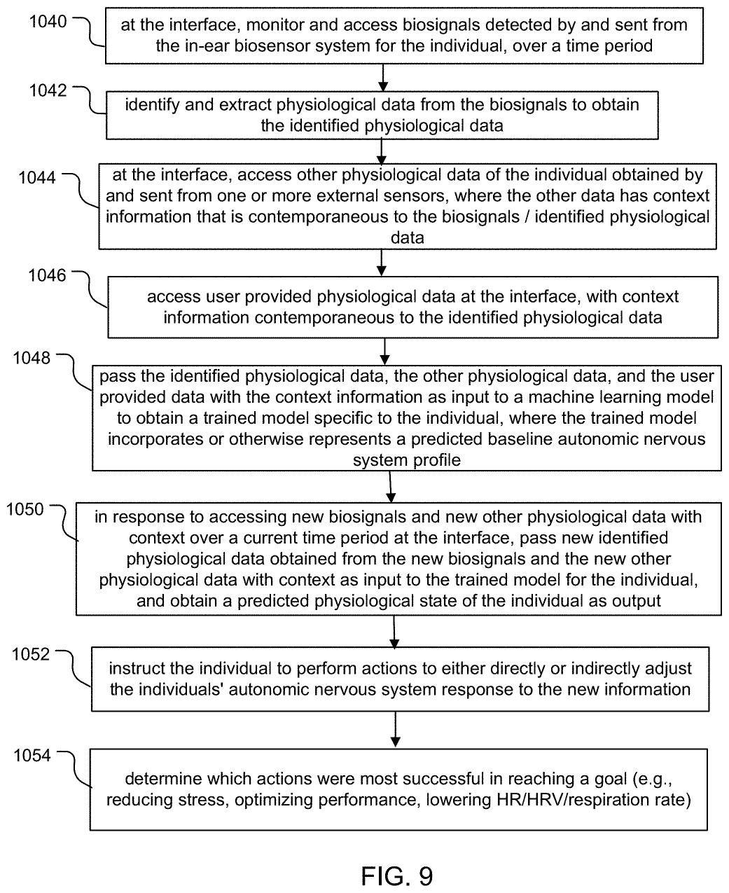

FIG. 9 is a flowchart that describes a biofeedback method of the closed loop system. Here, the method describes how the closed loop system 10 can receive information including the biosignals from the in-ear biosensor system 102 in conjunction with other physiological data obtained by and sent from external sensors/external systems, and execute actions based upon the information to improve the individual's health.

The physiological data 410 has common traits across individuals, but each individual can experience and react to the same stimuli differently. Not only can aspects of the biosignals 101 and the behavioral data vary among different individuals, based on factors such as age, sex, racial/ethnic group, life experience and educational background, but each individual 101 may behave or react different differently to the same stimulus at different times in their lives. For this reason, the closed loop system 10 obtains and stores multiple time-stamped instances of biosignals 101 and physiological data 410 for each individual 100.

In step 1040, at the interface 134, the data analysis system 109 monitors and accesses biosignals 101 detected by and sent from the in-ear biosensor system 102 for the individual 100, over a time period. In step 1042, the data analysis system 109 identifies and extracts physiological data 410 from the biosignals 101 to obtain the identified physiological data.

At step 1044, at the interface, the data analysis system 109 accesses other physiological data 410 of the individual 100 obtained by and sent from one or more external sensors, where the other data has context information that is contemporaneous to the biosignals/identified physiological data. The external data might be pupil diameter data 410-12 sent from the VR headset 58 or augmented reality device, from the eyeglass device 107-2, body temperature data 410-11 sent from an external temperature sensor/wearable 38, and humidity data of a room sent from an external humidity sensor, in examples. Additionally or alternatively, one or more of the other physiological data 410 might be detected by and sent to the interface 134 from one or more auxiliary sensors included within the earbuds 103 of the in-ear biosensor system 102.

Typically, the other physiological data is contemporaneous to the biosignals/identified physiological data, by virtue of the fact that the various systems or sensors that detect the other data either assign time stamps to the data/include metadata with the other data, or the interface 134 assigns time stamps to the received data. In this way, the data analysis system 109 can synchronize the time-stamped physiological data (identified from the time-stamped biosignals 101) with the time-stamped other physiological data.

According to step 1046, the data analysis system 109 might also access user provided physiological data at the interface 134, with context information contemporaneous to the identified physiological data 410. In examples, the user provided physiological data can include: information indicating that the individual 100 is feeling dizzy, sweaty or is experiencing chest or arm pain, stomach ache, tiredness, anxiety, stress or fear, in examples. For this purpose, the individual 100 might enter this information via the app 40 of the user device 107-1, or a medical professional 110 might provide this information to the interface 134 on behalf of the individual 100, in examples. The app 40 or other system might time-stamp the information sent to the interface 134, or the interface 134 might provide this function.

In other examples, the other physiological data can be eye movement and facial features obtained by the camera 98 of the eyeglass user device 107-2, or other camera; input provided by the individual 100 at an interactive video game, military training video, or virtual reality session; rapid body movements or shouting/screaming detected during real or simulated “fight or flight” scenarios; and various physical behaviors detected in response to other sensory stimuli. These stimuli can include: loud sounds, gunshot sounds, and soothing sounds and tones; unpleasant and pleasant smells or odors; reactions to changes in external pressure, heat and cold, brightness and darkness, in examples.

In still other examples, relative lack of behavioral data in response to stimuli can also be obtained. For example, minimal movement or change in behavior of an individual in response to a stimuli that most people would consider extremely stressful may also be an important behavioral characteristic. Such a behavioral response might be an early indicator of depression or stress, or problems interacting socially with others.

According to step 1048, the data analysis system 109 passes the identified physiological data, the other physiological data, and the user provided data with the context information as input to a machine learning model to obtain a trained model specific to the individual. The trained model incorporates or otherwise represents a predicted baseline autonomic nervous system profile 502.

Then, in step 1050, the data analysis system 109 accesses new biosignals 101 and new other physiological data at the interface 134, for a current time period. The data analysis system 109 identifies new physiological data 410 from the biosignals 101, passes the new identified physiological data and the new other physiological data with context as input to the trained model for the individual 100. The output of this operation is a predicted physiological state of the individual 100.

According to step 1052, the data analysis system 109 might instruct the individual 100 to perform actions to either directly or indirectly adjust the individuals' autonomic nervous system response to the new information. For this purpose, in examples, the data analysis system 109 might send instructions to the app 40 to present soothing audible tones to the individual via speakers of the earbuds 103, or instruct another application such as a music application (e.g., Spotify, Pandora, or the like) to play soothing music or aggressive, fast-paced music, depending on the desired target physiological state.

These actions might either directly or indirectly adjust the individuals' autonomic nervous system response to the new information. Direct actions can include: sending soothing tones to the speakers of the earbuds 103 to calm the individual 100 and adjust their HR, HRV and respiration; changing the scenery or environment of an interactive video game or VR training exercise to be less stressful; and sending commands to internet-enabled devices for changing lighting or heat in the room. Indirect actions can include: sending audio messages to the earbuds 103 that suggest or recommend breathing exercises or other ways for the individual 100 to train themselves to adjust their autonomic nervous system; presenting plots of the biosignals 101 at the display 88 of a user device 107, so that the individual 100 can see the changes to their physiological processes; or sending audio messages to the earbuds 103 or text messages to the user devices 107 for the individual 100 to manually carry out any of the direct actions.

In still other examples, rather than taking actions to adjust the individual's behavior in response to possibly stressful stimuli, the closed loop system 100 might also take actions to optimize the individual's performance on specific tasks. In one example, the actions might include indirect actions such as instructing military personnel that they are over-exerting on a specific task or expending too many calories, and that the completion of a mission might be jeopardized if they do not rest or slow down.

According to step 1054, the data analysis system 109 can determine or learn which actions were most successful in reaching a goal (e.g., reducing stress, optimizing performance, lowering HR/HRV/respiration rate), and update the personalized response profiles in response.

The closed loop system 10 has many applications. In examples, these applications include: performance training of athletes and military personnel, where the system may optimize the performance for a specific task or exercise, or emphasize performance across a larger scope (e.g. mission-based); stress and/or anxiety reduction; business performance and personal coaching; creation of personalized profiles for targeted advertising, such as within online social media platforms and in internet-based web search browsers and tools; gaming environments, including interactive and multi-player games conducted over public or private networks; and meditation and meditation training.

8 The closed loop system 10 also has advertising capabilities. For this purpose, the system 10 can send a user/subscriber individual 100 of the in-ear biosensor system 102 an advertisement, detect biosignals 101 from the user via the earbuds 103 and process the biosignals to determine one or more states of the individual in real-time. Here, the closed loop system 10 can collect and process biosignals prior to the presentation of the advertisement, during the time in which the individual perceives the advertisement, and afterward. The states may include states along a spectrum such as engaged or disengaged, pleased or annoyed, energetic or tired, or any number of scales for which biosignals can be indicative. Based on the state of the individual, the system 10 can determine whether to continue to show or otherwise present the original advertisement or switch to a different advertisement, in examples.

In examples, the advertisement could be in the form of a 2D or 3D experience and might be visual and/or audible in nature. In more detail, the advertisement might be in the form of placement of images or video frames of the product placed in view of the user, such as via the displays 88 of the user devices 107-2/107-2 and the VR headset 58, or the playing of a related sound, sequence of sounds, spoken description of the product, and possibly even music. The biosignals can include heart rate, heart rate variability, respiratory rate, and other biosignals that are deterministic of users' relative emotional state.

Other aspects of the closed loop system 10 are as follows. The system 10 can further refine the ability to select the advertisement sent to the user by collecting data about which advertisements cause which states, in another example. The closed loop system 10 can use this data in conjunction with data from other users to create associations between advertisements. The closed loop system 10 can then use the information regarding the associations to identify advertisements that were not yet shown. Such a capability can be effective for placing the user in a desired state based on known associations.

The in-ear biosensor system 102 is particularly effective because it incorporates the ability to detect the biosignals 101 and obtain information such as the physiological data 410 from the biosignals 101 within the same device. In contrast, many wearable systems require two wearables: a first wearable that detects some biosignals and creates a representation of the signals; and a second wearable that collects the representation of the signals sent from the first wearable. Typically, these wearable systems also process time-averaged versions of the signal representations, rather than processing the data in real-time. In contrast, the ability of the in-ear biosensor system 102 to obtain real-time physiological data 410 (versus averaged signals obtained by wearables) has advantages. In one example, the in-ear biosensor system 102 allows for a dramatic increase in efficiency and effectiveness. In the case of advertisements, in particular, the loss of a few seconds can result in a missed opportunity to present the advertisement at the optimal time.

The closed loop system might also access one or more stored and anonymized baseline autonomic nervous system profiles of other individuals. The data analysis system determines whether the baseline autonomic nervous system profiles of the other individuals are similar to the baseline autonomic nervous system profile of the individual, and can use the similar baseline autonomic nervous system profiles to predict changes to the current physiological state of the individual.

Additionally or alternatively, the closed loop system has music applications. For this purpose, the system 10 might create song lists to either calm down or energize individuals 100 based on their response, profiles 502 and predictions from other individuals 100 with similar baseline autonomic nervous system profiles 502 or application-specific profiles. In another example, the closed loop system 10 has work space applications. For this purpose, the system 10 might help people to obtain focus and performance based on whether they need to stay calm or be more alert, in examples.

In yet another example, the closed loop system 10 has social media and dating applications. For this purpose, the system 10 might influence the selection and/or matching of individuals as potential dating partners, potential parties to add to their list of people with whom they communicate or share mutual interests based on their autonomic nervous system profiles 502, in examples.

The closed loop system 10 can also present the current physiological state of the individual 100 and the baseline autonomic nervous system profile 502 of the individual to the interface 134. In this way, one or more external systems such as social media platforms and gaming system platforms can access the current physiological state of the individual and the baseline autonomic nervous system profile 502, and tailor application-information information that is based upon the current physiological state of the individual 100 and the baseline autonomic nervous system profile 502.

In another example, the data analysis system 109 might accesses a target physiological state at the interface 134 that was sent to the interface by a system external to the closed loop system. The closed loop system might then instruct the individual 100 to perform one or more actions designed to adjust the current physiological state of the individual to be that of the target physiological state. For example, prior to the individual taking a stressful standardized exam, an app provided ahead of time by the test administrators that is executing on the user device 107-1 might send instructions to the interface 134 for the individual 100 to transition to a more calm physiological state. The data analysis system 109 might read the instructions, and either suggest that the individual 100 engage in calming behavior (e.g., deep breathing exercises), or could select a song, nature sounds such as running water for playback by a music app executing on the user device 107-1.

While this invention has been particularly shown and described with references to preferred embodiments thereof, it will be understood by those skilled in the art that various changes in form and details may be made therein without departing from the scope of the invention encompassed by the appended claims.

We claim:

1. A closed loop system, the system comprising:
an interface configured to receive biosignals including infrasonic signals from an in-ear biosensor system worn by an individual; and
a data analysis system that monitors the received biosignals at the interface over time and identifies physiological data of the individual based upon the received biosignals;
wherein the data analysis system creates a baseline autonomic nervous system profile of the individual over a time period from the identified physiological data, and wherein the baseline autonomic nervous system profile tracks changes to a physiological state of the individual over the time period; and
wherein the data analysis system identifies current physiological data of the individual from new biosignals received at the interface over a current time period, and identifies a current physiological state of the individual by mapping the current identified physiological data against the baseline autonomic nervous system profile.

2. The closed loop system of claim 1, wherein the physiological data includes a heart rate, a heart rate variability, a blood pressure measurement, a respiration rate, a stroke volume and a heart contractility of the individual.

3. The closed loop system of claim 1, wherein the data analysis system creates a baseline autonomic nervous system profile over a time period by plotting one or more types of the identified physiological data against one or more other types of the physiological data.

4. The closed loop system of claim 1, wherein the data analysis system creates a baseline autonomic nervous system profile of the individual over a time period by passing the identified physiological data to a machine learning model for training, and wherein the trained machine learning model incorporates the baseline autonomic nervous system profile of the individual.

5. The closed loop system of claim 4, wherein the data analysis system maps the current identified physiological data against the baseline autonomic nervous system profile by passing the current identified physiological data as input to the trained machine learning model, the result of which is the current physiological state of the individual.

6. The closed loop system of claim 1, wherein the system creates the baseline autonomic nervous system profile of the individual from the identified physiological data and from other physiological data received at the interface, wherein the other physiological data is detected by and sent from one or more external sensors monitoring the individual.

7. The closed loop system of claim 1, wherein the system creates the baseline autonomic nervous system profile of the individual from the identified physiological data and from user provided physiological data received at the interface.

8. The closed loop system of claim 1, wherein the data analysis system presents the current physiological state of the individual and the baseline autonomic nervous system profile of the individual to the interface for access by one or more external systems.

9. The closed loop system of claim 1, wherein when the data analysis system maps the current identified physiological data against the baseline autonomic nervous system profile, if the current identified physiological data deviates from that of the physiological data in the profile by a threshold amount, the data analysis system instructs the individual to perform one or more actions designed to adjust the current physiological state of the individual to be similar to that of the physiological state in the profile.

10. The closed loop system of claim 1, wherein the data analysis system accesses a target physiological state at the interface that was sent to the interface by a system external to the closed loop system, and wherein the closed loop system instructs the individual to perform one or more actions designed to adjust the current physiological state of the individual to be that of the target physiological state.

11. A method of operation for a closed loop system, the method comprising:
(a) receiving, at an interface, biosignals including infrasonic signals from an in-ear biosensor system worn by an individual; and
(b) monitoring the received biosignals at the interface over time and identifying physiological data of the individual based upon the received biosignals;
(c) creating a baseline autonomic nervous system profile of the individual over a time period from the identified physiological data, the baseline autonomic nervous system profile tracking changes to a physiological state of the individual over the time period; and
(d) identifying current physiological data of the individual from new biosignals received at the interface over a current time period, and identifying a current physiological state of the individual by mapping the current identified physiological data against the baseline autonomic nervous system profile.

12. The method of claim 11, further comprising the physiological data including a heart rate, a heart rate variability, a blood pressure measurement, a respiration rate, a stroke volume and a heart contractility of the individual.

13. The method of claim 11, wherein the creating of (c) comprises creating a baseline autonomic nervous system profile over a time period by plotting one or more types of the identified physiological data against one or more other types of the physiological data.

14. The method of claim 11, wherein the creating of (c) comprises creating a baseline autonomic nervous system profile of the individual over a time period by passing the identified physiological data to a machine learning model for training, the trained machine learning model incorporating the baseline autonomic nervous system profile of the individual.

15. The method of claim 14, further comprising the data analysis system mapping the current identified physiological data against the baseline autonomic nervous system profile by passing the current identified physiological data as input to the trained machine learning model, the result of which is the current physiological state of the individual.

16. The method of claim 11, wherein the creating of (c) comprises creating the baseline autonomic nervous system profile of the individual from the identified physiological data and from other physiological data received at the interface, wherein the other physiological data is detected by and sent from one or more external sensors monitoring the individual.

17. The method of claim 11, wherein the creating of (c) comprises creating the baseline autonomic nervous system profile of the individual from the identified physiological data and from user provided physiological data received at the interface.

18. The method of claim 11, further comprising presenting the current physiological state of the individual and the baseline autonomic nervous system profile of the individual to the interface for access by one or more external systems.

19. The method of claim 11, wherein when mapping the current identified physiological data against the baseline autonomic nervous system profile, if the current identified physiological data deviates from that of the physiological data in the profile by a threshold amount, the method then instructing the individual to perform one or more actions designed to adjust the current physiological state of the individual to be similar to that of the physiological state in the profile.

20. The method of claim 11, further comprising accessing a target physiological state at the interface that was sent to the interface by a system external to the closed loop system, and instructing the individual to perform one or more actions designed to adjust the current physiological state of the individual to be that of the target physiological state.
